(19) United States
(12) Patent Application Publication
(10) Pub. No.: US 2004/0086984 A1
(43) Pub. Date: May 6, 2004

(54) Microbiological method for producing ascorbic acid

(76) Inventors: Clarence I. Kado, Davis, CA (US); Catherine J. Pujol, Santee, CA (US); Alexander W. Chan, Southbury, CT (US)

Correspondence Address: Paul D. Greeley, Esq., Ohlandt, Greeley, Ruggiero & Perle, L.L.P., One Landmark Square, 10th Floor, Stamford, CT, 06901-2682, US

(21) Appl. No.: 10/285,328

(22) Filed: Oct. 31, 2002

(51) Int. Cl. C12P 17/04; C12N 1/21; C12N 1/18

(52) U.S. Cl. 435/126; 435/252.3; 435/252.31; 435/252.33; 435/254.2

(57) ABSTRACT

There is provided a microbiological one-step method for the manufacture of ascorbic acid. The method comprises use of the operon expressing 2-ketogluconate dehydrogenase in naturally occurring microorganisms and microorganisms transformed with DNA encoding the 2-ketogluconate dehydrogenase gene. Ascorbic acid is obtained from culturing microorganisms expressing 2-ketogluconate dehydrogenase.

### BACKGROUND OF THE INVENTION

1. Field of the present invention

The present invention relates to a novel method for the production of ascorbic acid. More particularly, the invention relates to a one-step method of producing ascorbic acid by culture of microorganisms having 2-ketogluconate dehydrogenase.

2. Description of the Prior Art

Ascorbic acid, also known as vitamin C, is a dietary factor which must be present in the human diet to prevent scurvy, and which as an anti-oxidant has been identified as an agent that increases resistance to infection. Ascorbic acid is used commercially, for example as a nutritional supplement, color fixing agent, flavoring and preservative in meats and other foods, anti-oxidant in bread dough, abscission of citrus fruit in harvesting and as a reducing agent in analytical chemistry. Ascorbic acid also promotes human physiological functions such as the adsorption of iron, cold tolerance, the maintenance of the adrenal cortex, wound healing, the synthesis of polysaccharides and collagen, the formation of cartilage, dentine, bone and teeth, the maintenance of capillaries, and is useful as an antioxidant.

For use as a dietary supplement, ascorbic acid can be isolated from natural sources, such as rosehips, synthesized chemically through the two-step oxidation of L-sorbose, or produced by the oxidative fermentation of calcium D-gluconate by Acetobacter suboxidans. It is also known to obtain predominantly intracellular ascorbic acid through culture of the photosynthetic green alga Chlorella pyrenoidosa. It is believed that ascorbic acid is produced inside the chloroplasts of photosynthetic microorganisms and functions to neutralize energetic electrons produced during photosynthesis. Accordingly, ascorbic acid production is known in photosynthetic organisms as a protective mechanism. Additionally, ascorbic acid precursors and end products are known to occur in a catalogue of microorganisms, including recombinants.

The synthesis of ascorbic acid has received considerable attention over many years due to its relatively large market volume and high value as a specialty chemical. The Reichstein-Grussner method, a chemical route from glucose to ascorbic acid, was first disclosed in the mid-1930s. Over the years, advances in science and technology have led to numerous technical modifications that have improved the efficiency in the production of vitamin C and have been incorporated into the Reichstein-Grussner synthesis, a method used in industry today. Additionally, subsequent methods have included a bioconversion method for production of an intermediate of ascorbic acid, 2-keto-L-gulonic acid (2-KLG, KLG) which can be chemically converted to ascorbic acid. Also reported has been the construction of an expression system for the two-step production of 2-KLG from D-sorbitol. The presence of ascorbic acid in yeasts has been reported and the conversion of L-galactonic substrates to ascorbic acid in Candida yeast has been disclosed.

In spite of the scientific advances made in the production of ascorbic acid and its biocatalytic intermediates, there remains a need for methods for the production of ascorbic acid in order to supply the world's demand. Additionally, many of the methods utilized for the production or synthesis of ascorbic acid fail to be cost-effective so as to be commercially viable. The discovery of a new, cost-effective and relatively simple method for the production of ascorbic acid would be advantageous.

Accordingly, it is an object of the invention to provide a microbiological method for the production of ascorbic acid.

It is another object of the invention to provide a method for the production of ascorbic acid from microorganisms having 2-ketogluconate dehydrogenase.

It is yet another object of the invention to provide microorganisms having 2-ketogluconate dehydrogenase capable of producing ascorbic acid.

These and other objects and advantages of the present invention and equivalents thereof are achieved by methods and microorganisms described herein.

### SUMMARY OF THE INVENTION

The present invention provides a method of producing ascorbic acid from a microorganism encoding expressionable 2-ketogluconate dehydrogenase. The method comprises the steps of: culturing a microorganism encoding expressionable 2-ketogluconate dehydrogenase microorganism; and recovering ascorbic acid. Ascorbic acid is preferably secreted into liquid culture medium.

Preferred microorganisms for production of ascorbic acid are bacteria and fungi. Microbial culture is preferably under aerobic conditions in liquid culture medium comprising glucose as a carbon source using a batch culture system, a continuous culture system, a fixed bed culture system, or any combinations of these systems. A microorganism suitable for the production of ascorbic acid may be a naturally occurring microorganism, or mutant thereof, having the operon 2-ketoguconate dehydrogenase. Alternatively, a transformed microorganism encoding 2-ketogluconate dehydrogenase may be used. A transformed microorganism of the invention is preferably a bacterium or a fungus. A microorganism suitable for transformation is preferably a member of the following genera: Pantoea, Escherichia, Bacillus, Lactobacillus, Bifidobacterium, Brevibacterium, Xanthomonas, Streptococcus, Leuconostoc, and Saccharomyces. More preferably, a transformed microorganism of the invention is of P. citrea, E. coli, B. subtilis, L. lactis, S. cerevesiae, or mutants, varieties and strains thereof.

Culturing of a microorganism for the production of ascorbic acid is preferably aerobic, in liquid culture medium comprising physiologically acceptable amounts of organic and inorganic nutrients essential for growth of such microorganism, and at physiologically acceptable pH and temperature for a period of time sufficient to produce ascorbic acid. Culturing may be in a batch culture system, a continuous culture system, a fixed bed culture system, or any combination of such systems. Culture medium having glucose as a carbon source is preferred. Culturing is preferably at a temperature from about 20 to about 37° C., and in a medium where the pH is from about 3 to about 10.

An embodiment of the method of the invention for the manufacture of ascorbic acid comprises the steps of: transforming host cells with an expression vector containing DNA encoding 2-ketogluconate dehydogenase; culturing the transformed host cells; and recovering ascorbic acid. The ascorbic acid is preferably secreted into the culture medium. Transformed host cells are preferably a microorganism selected from the genera Pantoea, Escherichia, Bacillus, Lactobacillus, Bifidobcacterium, Brevibacterium, Streptococcus, Leuconostoc, Xanthomonas, and Saccharomyces. Transformed host cells are preferably either bacteria or fungi, and microbes are preferably cultured under aerobic conditions in batch, continuous, or fixed bed culture systems in a liquid medium. Culture media contain glucose as a preferred carbon source. Transformed host cells are preferably a microorganism of the following species: P. citrea, E. coli B. subtilis, L. lactis, X. campestris S. cerevesiae, and mutants, varieties and strains thereof. When transform ed host cells are a microorganism, culturing is preferably (i) under aerobic conditions; (ii) in a system of batch culture, continuous culture, fixed bed culture or any combinations thereof; (iii) in a liquid culture medium comprising physiologically acceptable amounts of organic and inorganic nutrients essential for growth of the microorganism; (iv) at physiologically acceptable pH and temperature; and (v) for a period of time sufficient to produce ascorbic acid. Culture medium for recombinant microorganisms preferably contains glucose as a carbon source. Preferably, culture of microorganisms is at a temperature from about 20 to about 37° C. and at a pH from about 3 to about 10. Preferably, ascorbic acid is secreted into the culture medium.

As an embodiment of the invention, a recombinant microorganism for producing ascorbic acid may be prepared by a process comprising the steps of: selecting a microorganism from genera of the group of Pantoea, Escherichia, Bacillus, Lactobacillus, Streptococcus, Leuconostoc, Xanthomonas, Brevibacterium and Saccharomyces; transforming the microorganism with an expression vector containing DNA encoding 2-ketogluconate dehydrogenase, where the resulting transformed microorganism is capable of expressing 2-ketogluconate dehydrogenase and producing ascorbic acid. Preferred microorganism of the invention are Pantoea citrea, Escherichia coli, Bacillus subtilis, Lactobacillus lactis and related species, e.g., Lactobacillus delbrueckii, Streptococcus lactis, Xanthomonas campestris, Saaccromyces cerevesiae, and mutants, varieties and strains thereof.

### BRIEF DESCRIPTION OF THE DRAWINGS

FIG. 1 is a graph showing Vitamin C (i.e., ascorbic acid) production utilizing Pantoea citrea 1056R; and

FIG. 2 is a graph showing the standard curve for ascorbic acid production according to the present invention.

### DETAILED DESCRIPTION OF THE INVENTION

Nucleic acid encoding 2-ketogluconate dehydrogenase (2KGADH). The novel DNA for encoding 2KGADH from P. citrea has been isolated and sequenced. The 2KGADH operon and the technique for isolating and sequencing such DNA is described in detail in Pujol et al., [J. Bacteriology, 182:2230-2237 (2000)], and is incorporated by reference into this disclosure in its totality.

The nucleotide sequence of the novel DNA isolated from P. citrea has been filed in the GenBank database, under Accession No. AF131202, and this sequence of DNA (encoding the operon of 2-ketogluconate dehydrogenase including the genes orfB, orfC and orfA) is incorporated by reference in its entirety into this disclosure. As disclosed in Pujol et al., supra, the novel DNA is a 4349 bp sequence comprising three genes, identified as orfB, orfC and orfA. These genes are approximately 569 bp, 1661 bp and 1073 bp, in length, respectively.

The isolated DNA encodes for a 2KGADH complex, comprising three subunits, derived from the three genes: 1) a flavoproteih dehydrogenase of between approximately 492 to 553 amino acids (encoded by orfB); 2) an associated cytochrome C of between approximately 357 to 396 amirio acids (encoded by orfc); and 3) a subunit of unknown function of approximately 189 amino acids (encoded by orfA). These subunits have all been characterized and their amino acid sequences determined (c.f., Pujol et al., supra).

The three subunits show percent (%) similarities with corresponding proteins from other bacteria. The orfB gene protein shares a 34% similarity with an Erwinia cypripedii membrane-bound gluconate dehydrogenase. The orfC gene protein shares a 60% similarity to the cytochrome c subunit of an E cypripedii gluconate dehydrogenase complex, as well as to the alcohol dehydrogenase of A. aceti.

The present invention provides a method for the use of the isolated gene encoding a membrane-bound 2KGADH from P. citrea. A recombinant plasmid is conveniently prepared by recombinant DNA technology containing the isolated DNA encoding 2KGADH. A suitable microbial host cell is conveniently transformed with a recombinant plasmid containing the isolated DNA encoding the 2KGADH. 2,5-diketogluconic acid (2,5-KDG) is produced from 2-ketogluconic acid by culturing the transformed cell in glucose containing medium. Ascorbic acid is produced from 2,5KDG as a desired end product from the recombinant cell culture. Recombinant DNA technology as used herein refers to technology for transformation of microorganism well known in the art.

The novel DNA encoding the 2KGADH operon and the proteins for which for it encodes, have a noted utility for producing ascorbic acid. Ascorbic acid, commonly referred to as Vitamin C, is often added to foodstuffs as a nutritional supplement or else is sold in pure form as Vitamin C tablets.

Sonoyama et al. (1987, Agric. Biol. Chem. 51:2003-2004) and Anderson et al. (1985, Science 230:144-149) disclose ascorbic acid precursors and end products occurring in a catalogue of microorganisms, including recombinants. These references do not cite P. citrea as one of the microorganisms capable of ascorbic acid production.

In the cell metabolism of P. citrea, it has been determined that 2-ketogluconate is converted by 2KGADH to produce 2,5-diketogluconate, part of which is converted to ascorbic acid. This discovery of ascorbic acid production from P. citrea is believed to be the first such discovery from this bacterial strain.

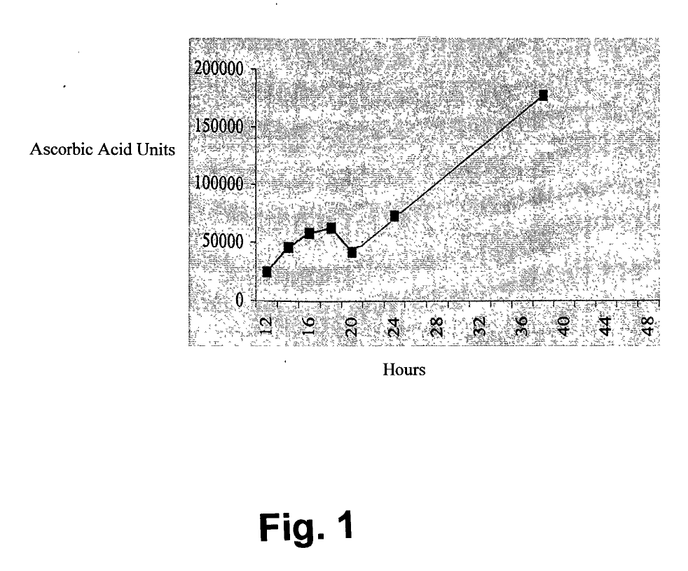

FIG. 1 shows the production over time results of ascorbic acid by P. citrea. As shown in FIG. 1, approximately 180,000 units of ascorbic acid are produced at 37 hours incubation at 28 degrees Centigrade. This amount of ascorbic acid is equivalent to about 16 milligram per milliliter yield of ascorbic acid.

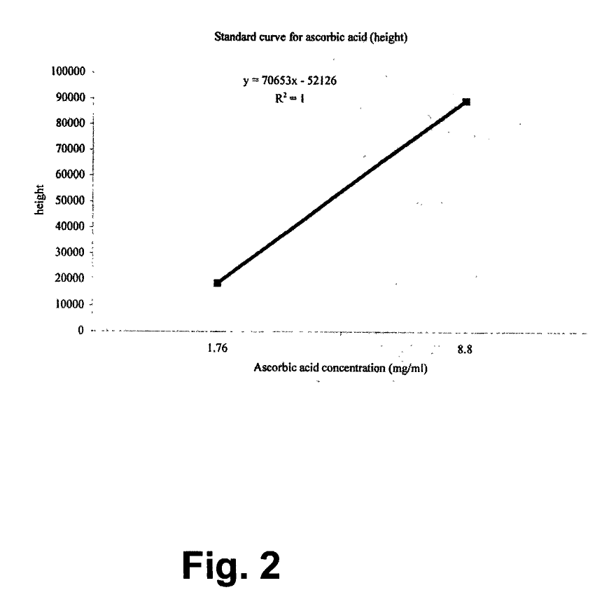

FIG. 2 shows the standard curve for ascorbic acid production.

This discovery of ascorbic acid from P. citrea lends to an important utility of the novel DNA and 2KGADH complex for which it encodes. The novel DNA, which has been isolated and sequenced, can be cloned into an appropriate bacterial expression system wherein ascorbic acid production can be optimized. Suitable microorganisms for creating recombinants containing the novel DNA include P. citrea (various strains), Escherichia coli, Bacillus subtilis, Xanthomonas campestris, Lactobacillus lactis, and Saccharomyces cerevesiae. These bacteria and fungus are believed to represent suitable expression systems for ascorbic acid production using the P. citrea gene.

2KGADH Expression. GenBank database Accession No. AF102175, incorporated herein in its totality by reference, provides the nucleotide sequence for the Pantoea citrea thio-disulfide interchange protein (dsbD) gene. This nucleotide sequence is for an isolated DNA which includes the dsbD gene encoding a P. citrea thiol-disulfide interchange protein, a protein involved in the biogenesis of c-type cytochromes. GenBank database Accession No. AF 103874, incorporated in its totality by reference, discloses a nucleotide sequence for an isolated DNA which includes an eight-gene operon (ccmA, ccmB, ccmC, ccmD, ccmE, ccmF, ccmG, ccmH) encoding for eight proteins, of which one or more appeared to be required for cytochrome c maturation.

Of these, the DsbD and CcmC proteins were demonstrated to solely affect the biogenesis and maturation of c-type cytochromes, such as the cytochrome c subunit of 2KGADH. This affect demonstrates that the protein products of the dsbD and ccmC genes have a utility in regulating the operation of the 2KGADH complex. The regulating quality of these proteins could be put to potential use in regulating the ascorbic acid production of any bacterial expression system containing the novel DNA for encoding the 2KGADH protein.

Upon isolating and sequencing, the DsbD protein of P. citrea was found to be 80% similar to a corresponding DsbD protein of E. coli. Additionally, the CcmC protein of P. citrea was found to be 82% similar to a corresponding CcmC protein of E. coli.

Culturing microorganism of the invention having the 2KGADH operon preferably secrete ascorbic acid into the culture medium. Ascorbic acid may conveniently be recovered by techniques known in the art. For example, with batch culture, cells may be separated by filtration, sedimentation, centrifugation or other known methodology. Culture filtrate or supernatant as specified by the methodology, may, as necessary be further treated (i.e., precipitation of protein, exocellular material etc.) and ascorbic acid thereafter recovered. Suitable standardized culture media for microbial culture containing glucose should be employed. Essential organic and inorganic nutrients are provided by the culture medium. Culture conditions which are ordinary in the art are employed (i.e., agitation, pH, temperature, etc) and are preferably optimized.

The foregoing disclosure of genetic sequences, products and methods is considered as only illustrative of the preferred, embodiments of, and not a limitation upon, the scope of the invention. Although the present invention describes in detail certain embodiments, it is understood that variations and modifications exist known to those skilled in the art that are within the invention. Accordingly, the present invention is intended to encompass all such alternatives, modifications and variations that are within the scope of the invention as set forth in the following claims.

What is claimed is:

1. A method of producing ascorbic acid from a microorganism encoding expressionable 2-ketogluconate dehydrogenase comprising the steps of:
culturing said microorganism; and
recovering ascorbic acid.

2. The method of claim 1, wherein said microorganism is selected from the group of bacteria and fungi.

3. The method of claim 1, wherein said microorganism is a bacterium and said culturing is under aerobic conditions in liquid culture medium comprising glucose as a carbon source in a culture system selected from the group of batch, continuous, fixed bed, and any combinations thereof.

4. The method of claim 1, wherein said microorganism is a naturally occurring microorganism or mutant thereof comprising 2-ketoguconate dehydrogenase.

5. The method of claim 1, wherein said microorganism encoding 2-ketogluconate dehydrogenase is a transformed microorganism.

6. The method of claim 5, wherein said wherein said transformed microorganism is a bacterium or fungus.

7. The method of claim 5, wherein said transformed microorganism is selected from a genus of the group of Pantoea, Escherichia, Bacillus, Lactobacillus, Xanthomonas, Brevibacterium, Bifidobacterium, Streptococcus, Leuconostoc and Saccharomyces.

8. The method of claim 7, wherein said transformed microorganism is selected form the group of P. citrea, E. coli, B. subtilis, L. lactis, S. cerevesiae, and mutants, varieties and strains thereof.

9. The method of claim 1, wherein said culturing of said microorganism is under aerobic conditions, in liquid culture medium comprising physiologically acceptable amounts of organic and inorganic nutrients essential for growth of said microorganism, and at physiologically acceptable pH and temperature for a period of time sufficient to produce said ascorbic acid in a culture system selected from the group of batch culture, continuous culture, fixed bed culture, and any combinations thereof.

10. The method of claim 9, wherein said culture medium comprises glucose as a carbon source.

11. The method of claim 9, wherein said temperature is from about 20 to about 37° C., and said pH is from about 3 to about 10.

12. A method of producing ascorbic acid comprising the steps of:
transforming host cells with an expression vector containing DNA encoding 2-ketogluconate dehydogenase;
culturing said transformed host cells; and
recovering ascorbic acid.

13. The method of claim 12, wherein said transformed host cells are a microorganism selected from a genus of the group of Pantoea, Escherichia, Bacillus, Lactobacillus, Xanthomonas, Brevibacterium, Bifidobacterium, Streptococcus, Leuconostoc and Saccharomyces.

14. The method of claim 12, wherein said transformed host cells are selected from the group of bacteria and fungi, and said culturing is under aerobic batch, continuous, or fixed bed culture conditions in a liquid medium.

15. The method of claim 12, wherein said transformed host cells are a microorganism selected from the group of P. citrea, E. coli, B. subtilis, L. lactis, X. campestris, S. lactis, S. cerevesiae, and mutants, varieties and strains thereof.

16. The method of claim 12, wherein said transformed host cells are a microorganism, said culturing is aerobic; in a system selected from the group of batch culture, continuous culture, fixed bed culture and any combinations thereof; in a liquid culture medium comprising physiologically acceptable amounts of organic and inorganic nutrients essential for growth of said microorganism; and said culturing is at physiologically acceptable pH and temperature for a period of time sufficient to produce said ascorbic acid.

17. The method of claim 16, wherein said culture medium comprises glucose as a carbon source.

18. The method of claim 16, wherein said temperature is from about 20 to about 37° C. and said pH is from about 3 to about 10.

19. A recombinant microorganism for producing ascorbic acid, said microorganism prepared by a process comprising the steps of:
selecting a microorganism from genera of the group of Pantoea, Escherichia, Bacillus, Xanthomonas, Brevibacterium, Bifidobacterium, Leuconostoc, Streptococcus, Lactobacillus, and Saccharomyces;
transforming said microorganism with an expression vector containing DNA encoding 2-ketogluconate dehydrogenase, wherein said transformed microorganism is capable of expressing 2-ketogluconate dehydrogenase and producing ascorbic acid.

20. A microorganism according to claim 19, wherein said microorganism is selected form the group of Pantoea citrea, Escherichia coli, Bacillus subtilis, Lactobacillus lactis, Lactobacillus delbruckii, Streptococcus lactis, Saccharomyces cerevesiae, and mutants, varieties and strains thereof.
